(19) United States
(12) Patent Application Publication
(10) Pub. No.: US 2005/0009873 A1
(43) Pub. Date: Jan. 13, 2005

(54) Acyl dihydro pyrrole derivatives as hcv inhibitors

(76) Inventors: Gianpaolo Bravi, Stevenage (GB); Peter David Howes, Stevenage (GB); Victoria Lucy Helen Lovegrove, Stevenage (GB); Pritom Shah, Stevenage (GB); Martin John Slater, Stevenage (DE)

Correspondence Address: DAVID J LEVY, CORPORATE INTELLECTUAL PROPERTY;GLAXOSMITHKLINE, FIVE MOORE DR., PO BOX 13398, RESEARCH TRIANGLE PARK, NC, 27709-3398, US

(21) Appl. No.: 10/494,121

(22) PCT Filed: Oct. 30, 2002
(86) PCT No.: PCT/EP02/12171

(30) Foreign Application Priority Data
Nov. 2, 2001 (GB) 0126435.7
May 8, 2002 (GB) 0210513.8

(51) Int. Cl. C 07D 4 3/02; A61K 31/454; A61K 31/4025; A61K 31/401

(52) U.S. Cl. 514/326; 514/422; 514/423; 546/208; 548/517; 548/531

(57) ABSTRACT

Novel anti-viral agents of Formula (I) 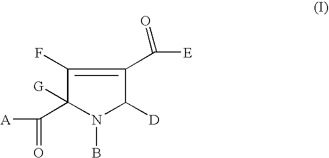 wherein:
A represents OR¹, NR¹R², or R¹ wherein R¹ and R² are hydrogen, C₁₋₆alkyl, aryl, heteroaryl, arylalkyl, or heteroarylalkyl; or R¹ and R² together with the nitrogen atom to which they are attached form a 5 or 6 membered saturated cyclic group;
B represents C(O)R³ wherein R³ is C₁₋₆alkyl, aryl, heteroaryl, arylalkyl, or heteroarylalkyl;
D represents C₁₋₆alkyl, aryl, heteroaryl or heterocyclyl;
E represents OR¹, NR¹R², or R¹ wherein R¹ and R² are hydrogen, C₁₋₆alkyl, aryl, heteroaryl, arylalkyl or heteroarylalkyl; or R¹ and R² together with the nitrogen atom to which they are attached form a 5 or 6 membered saturated cyclic group;
F represents hydrogen, C₁₋₆alkyl, aryl or heteroaryl; and
G represents hydrogen, C₁₋₆alkyl, heterocyclylalkyl, arylalkyl or heteroarylalkyl; and salts and solvates thereof, processes for their preparation and methods of using them in HCV treatment are provided.

### FIELD OF THE INVENTION

The present invention relates to novel acyl dihydro pyrrole derivatives useful as anti-viral agents. Specifically, the present invention involves novel HCV inhibitors.

### BACKGROUND OF THE INVENTION

Infection with HCV is a major cause of human liver disease throughout the world. In the US, an estimated 4.5 million Americans are chronically infected with HCV. Although only 30% of acute infections are symptomatic, greater than 85% of infected individuals develop chronic, persistent infection. Treatment costs for HCV infection have been estimated at $5.46 billion for the US in 1997. Worldwide over 200 million people are estimated to be infected chronically. HCV infection is responsible for 40-60% of all chronic liver disease and 30% of all liver transplants. Chronic HCV infection accounts for 30% of all cirrhosis, end-stage liver disease, and liver cancer in the U.S. The CDC estimates that the number of deaths due to HCV will minimally increase to 38,000/year by the year 2010.

Due to the high degree of variability in the viral surface antigens, existence of multiple viral genotypes, and demonstrated specificity of immunity, the development of a successful vaccine in the near future is unlikely. Alpha-interferon (alone or in combination with ribavirin) has been widely used since its approval for treatment of chronic HCV infection. However, adverse side effects are commonly associated with this treatment: flu-like symptoms, leukopenia, thrombocytopenia, depression from interferon, as well as anemia induced by ribavirin (Lindsay, K. L. (1997) Hepatology 26 (suppl 1):71S-77S). This therapy remains less effective against infections caused by HCV genotype 1 (which constitutes ˜75% of all HCV infections in the developed markets) compared to infections caused by the other 5 major HCV genotypes. Unfortunately, only ˜50-80% of the patients respond to this treatment (measured by a reduction in serum HCV RNA levels and normalization of liver enzymes) and, of those treated, 50-70% relapse within 6 months of cessation of treatment. Recently, with the introduction of pegylated interferon, both initial and sustained response rates have improved substantially, and combination treatment of Peg-IFN with ribavirin constitutes the gold standard for therapy. However, the side effects associated with combination therapy and the impaired response in patients with genotype 1 present opportunities for improvement in the management of this disease.

First identified by molecular cloning in 1989 (Choo, Q-L et al (1989) Science 244:359-362), hepatitis C virus (HCV) is now widely accepted as the most common causative agent of post-transfusion non A, non-B hepatitis (NANBH) (Kuo, G et al (1989) Science 244:362-364).

Due to its genome structure and sequence homology, this virus was assigned as a new genus in the Flaviviridae family. Like the other members of the Flaviviridae, such as flaviviruses (e.g. yellow fever virus and Dengue virus types 1-4) and pestiviruses (e.g. bovine viral diarrhea virus, border disease virus, and classic swine fever virus) (Choo, Q-L et al (1989) Science 244:359-3; Miller, R. H. and R. H. Purcell (1990) Proc. Natl. Acad. Sci. USA 87:2057-2061), HCV is an enveloped virus containing a single strand RNA molecule of positive polarity. The HCV genome is approximately 9.6 kilobases (kb) with a long, highly conserved, noncapped 5′ nontranslated region (NTR) of approximately 340 bases which functions as an internal ribosome entry site (IRES) (Wang CY et al ‘An RNA pseudoknot is an essential structural element of the internal ribosome entry site located within the hepatitis C virus 5′ noncoding region’ RNA—A Publication of the RNA Society; 1(5):526-537, July 1995). This element is followed by a region which encodes a single long open reading frame (ORF) encoding a polypeptide of ˜3000 amino acids comprising both the structural and nonstructural viral proteins.

Upon entry into the cytoplasm of the cell, this RNA is directly translated into a polypeptide of ˜3000 amino acids comprising both the structural and nonstructural viral proteins. This large polypeptide is subsequently processed into the individual structural and nonstructural proteins by a combination of host and virally-encoded proteinases (Rice, C. M. (1996) in B. N. Fields, D. M. Knipe and P. M. Howley (eds) Virology 2^{nd} Edition, p931-960; Raven Press, N.Y.). Following the termination codon at the end of the long ORF, there is a 3′ NOR which roughly consists of three regions: an ˜40 base region which is poorly conserved among various genotypes, a variable length poly(U)/polypyrimidine tract, and a highly conserved 98 base element also called the “3′ X-tail” (Kolykhalov, A. et al (1996) J. Virology 70:3363-3371; Tanaka, T. et al (1995) Biochem Biophys. Res. Conmmun. 215:744-749; Tanaka, T. et al (1996) J. Virology 70:3307-3312; Yamada, N. et al (1996) Virology 223:255-261). The 3′ NIR is predicted to form a stable secondary structure which is essential for HCV growth in chimps and is believed to function in the initiation and regulation of viral RNA replication.

The NSSB protein (591 amino acids, 65 kDa) of HCV (Behrens, S. E. et al (1996) EMBO J. 15:12-22), encodes an RNA-dependent RNA polymerase (RdRp) activity and contains canonical motifs present in other RNA viral polymerases. The NS5B protein is fairly well conserved both intra-typically (˜95-98% amino acid (aa) identity across 1b isolates) and inter-typically (˜85% aa identity between genotype 1a and 1b isolates). The essentiality of the HCV NS5B RdRp activity for the generation of infectious progeny virions has been formally proven in chimpanzees (A. A. Kolykhalov et aL. (2000) Journal of Virology, 74(4), p.2046-2051). Thus, inhibition of NS5B RdRp activity (inhibition of RNA replication) is predicted to cure HCV infection.

Based on the foregoing, there exists a significant need to identify synthetic or biological compounds for their ability to inhibit HCV.

### SUMMARY OF THE INVENTION

The present invention involves compounds represented hereinbelow, pharmaceutical compositions comprising such compounds and use of the compounds in treating viral infection, especially HCV infection.

### DETAILED DESCRIPTION OF THE INVENTION

The present invention provides compounds of Formula (I): 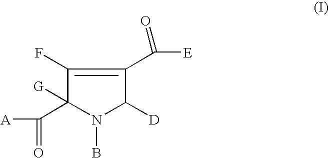
wherein:
A represents OR¹, NR¹R², or R¹ wherein R¹ and R² are independently selected from the group consisting of hydrogen, C₁₋₆alkyl, aryl, heteroaryl, arylalkyl, and heteroarylalkyl; or R¹ and R² together with the nitrogen atom to which they are attached form a 5 or 6 membered saturated cyclic group;
B represents C(O)R³ wherein R³ is selected from the group consisting of C₁₋₆alkyl, aryl, heteroaryl, arylalkyl, and heteroarylalkyl;
D represents C₁₋₆alkyl, aryl, heteroaryl or heterocyclyl;
E represents OR¹, NR¹R², or R¹ wherein R¹ and R² are independently selected from the group consisting of hydrogen, C₁₋₆alkyl, aryl, heteroaryl arylalkyl or heteroarylalkyl; or R¹ and R² together with the nitrogen atom to which they are attached form a 5 or 6 membered saturated cyclic group;
F represents hydrogen, C₁₋₆alkyl, aryl or heteroaryl; and
G represents hydrogen, C₁₋₆ alkyl, heterocyclylalkyl, arylalkyl or heteroarylalkyl; and salts, solvates and esters thereof, provided that when A is OR¹ then R¹ is other than tert-butyl.

As used herein, “alkyl” refers to an optionally substituted hydrocarbon group. The alkyl hydrocarbon group may be linear, branched or cyclic, saturated or unsaturated. Where the alkyl hydrocarbon group is cyclic, it will be understood that there will be a minimum of 3 carbon atoms in the group. Preferably, the group is saturated. Preferred alkyl moieties are C₁₋₄alkyl. Optional subsituents include C₁₋₆alkyl, halo, OR⁴, C(O)NR⁵R⁶, C(O)R³, CO₂H, CO₂R³, NR⁵R⁶, NHC(O)R³, NHCO₂R³, NHC(O)NR¹R², SO₂NR¹R², SO₂R³, nitro, cyano, oxo, and heterocyclyl. More preferably, the optional subsituents include C₁₋₆alkyl, halo, OR⁴, C(O)NR⁵R⁶, CO₂R³, NR⁵R⁶, NHC(O)R³, NHCO₂R³, NHC(O)NR¹R², SO₂NR¹R², SO₂R³, nitro, oxo, and heterocyclyl.

R⁴ represents hydrogen, C₁₋₆alkyl, arylalkyl, or heteroarylalkyl; R⁵ and R⁶ are independently selected from hydrogen, C₁₋₆alkyl, aryl and heteroaryl.

As used herein, “aryl” refers to an optionally substituted aromatic group with at least one ring having a conjugated pi-electron system, containing up to two conjugated or fused ring systems. “Aryl” includes carbocyclic aryl and biaryl groups, all of which may be optionally substituted. Preferred “aryl” moieties are unsubstituted, monosubstituted, disubstituted or trisubstituted phenyl. Preferred “aryl” substituents are selected from the group consisting of C₁₋₆alkyl, halo, OR⁴, C(O)NR⁵R⁶, C(O)R³, CO₂H, CO₂R³, NR⁵R⁶, NHC(O)R³, NHCO₂R³, NHC(O)NR¹R², SO₂NR¹R², SO₂R³, nitro, cyano, oxo, heterocyclyl, CF₃, pyridine, phenyl, and NO₂. More preferably, “aryl” substituents are selected from the group consisting of C₁₋₆alkyl, halo, OR⁴, C(O)NR⁵R⁶, CO₂R³, NR⁵R⁶, NHC(O)R³, NHCO₂R³, NHC(O)NR¹R², SO₂NR¹R², SO₂R³, nitro, oxo, heterocyclyl, OC₁₋₄alkyl, CF₃, pyridine, phenyl, and NO₂.

As used herein, “heteroaryl” refers to an optionally substituted, 5 or 6 membered, aromatic group comprising one to four heteroatoms selected from N, O and S, with at least one ring having a conjugated pi-electron system, containing up to two conjugated or fused ring systems. Preferred “heteroaryl” moieties are unsubstituted, monosubstituted, disubstituted or trisubstituted thienyl and thiazolyl. Preferred “heteroaryl” substituents are selected from the group consisting of C₁₋₆alkyl, halo, OR⁸, C(O)NR⁶R⁷, C(O)R³, CO₂H, CO₂R³, NR⁶R⁷, NHC(O)R³, NHCO₂R³, NHC(O)NR¹R², SO₂NR¹R², SO₂R³, nitro, cyano, oxo, heterocyclyl, CF₃, pyridine, phenyl, and NO₂. More preferred “heteroaryl” substituents are selected from the group consisting of C₁₋₆alkyl, halo, OR⁴, C(O)NR⁵⁶, CO₂R³, NR⁵R⁶, NHC(O)R³, NHCO₂R³, NHC(O)NR¹R², SO₂NR¹R², SO₂R³, nitro, oxo, heterocyclyl, OC₁₋₄alkyl, CF₃, pyridine, phenyl, and NO₂.

As used herein, “heterocyclic” and “heterocyclyl” refer to an optionally substituted, 5 or 6 membered, saturated cyclic hydrocarbon group containing one to four, preferably 1 or 2, heteroatoms selected from N, optionally substituted by hydrogen, C₁₋₆alkyl, C(O)R³, SO₂R³, aryl or heteroaryl; O; and S, optionally substituted by one or two oxygen atoms.

It will be appreciated that the compounds of the present invention may contain one or more asymmetric carbon atoms and may exist in racemic and optically active forms. All of these compounds and diastereoisomers are contemplated to be within the scope of the present invention.

Preferably, A is OR¹ where R¹ is hydrogen;

Preferably, when B represents C(O)R³, R³ is aryl or heteroaryl; more preferably, R³ is phenyl; especially preferred is R³ represents phenyl substituted at least in the para-position by tert-butyl; most preferred is R³ represents phenyl substituted in the para-position by tert-butyl and optionally further substituted, preferably meta-substituted, by methyl, ethyl, methoxy, ethoxy, or halo, preferably halo;

Preferably, D is selected from the group consisting of C₁₋₆alkyl, aryl and heteroaryl; more preferably D is heteroaryl; most preferably D is 1,3-thiazolyl;

Preferably, E is OR¹ where R¹ is hydrogen; or NR¹R² where R¹ and R² are independently selected from H, C₁₋₆alkyl, or arylalkyl;

Preferably, F is hydrogen or C₁₋₆alkyl; more preferably F is hydrogen;

Preferably, G is selected from the group consisting of C₁₋₆alkyl, arylalkyl and heteroarylalkyl; more preferably G is C₁₋₆alkyl.

It is to be understood that the present invention covers all combinations of suitable, convenient and preferred groups described herein.

Preferred compounds useful in the present invention are selected from the group consisting of:
rel-(2S,5R)-1-(4-tert-butylbenzoyl)-2-isobutyl-5-(1,3-thiazol-2-yl)-2,5-dihydro-1H-pyrrole-2,4dicarboxylic acid;
rel-(2S,5R)-1-(4-tert-butylbenzoyl)-2-isobutyl-3-methyl-5-(1,3-thiazol-2-yl)-2,5-dihydro-1H-pyrrole-2,4-dicarboxylic acid;
rel-(2S,5R)-1-(4-tert-butylbenzoyl)-4carbamoyl-2-isobutyl-5-(1,3-thiazol-2,5-dihydro-1H-pyrrole-2-carboxylic acid;
rel-(2S,5R)-1-(4-tert-butylbenzoyl)-4{[(-2-carboxyethyl)amino]carbonyl}-2-isobutyl-5-(1,3-thiazol-2-yl)-2,5-dihydro-1H-pyrrole-2-carboxylic acid;
rel-(2S,5R)-1-(4-tert-butylbenzoyl)-4-{[(3-carboxamidoethyl)amino]carbonyl}-2-isobutyl-5-(1,3-thiazol-2-yl)-2,5dihydro-1H-pyrrole-2-carboxylic acid;
rel-(2S,5R)-1-(4-tert-butylbenzoyl)-4-{[(2-carboxymethyl)amino]carbonyl}-2-isobutyl-5-(1,3-thiazol-2-yl)-2,5-dihydro-1H-pyrrole-2-carboxylic acid;
rel-(2S,5R)-1-(4-tert-butylbenzoyl)-4[(isobutylamino)carbonyl]-2-isobutyl-5-(1,3-thiazol-2-yl)-2,5-dihydro-1H-pyrrole-2-carboxylic acid;
rel-(2S,5R)-1-(4-tert-butylbenzoyl)-4-[(benzylamino)carbonyl]-2-isobutyl-5-(1,3-thiazol-2-yl)-2,5-dihydro-1H-pyrrole-2-carboxylic acid;
rel-(2S,5R)-1-(4-tert-butylbenzoyl)-4-{[(cyclohexylmethyl)amino]carbonyl}-2-isobutyl-5-(1,3-thiazol-2-yl)-2,5dihydro-1H-pyrrole-2-carboxylic acid;
rel-(2S,5R)-1-(4-tert-butylbenzoyl)-4-{[(cyanomethyl)amino]carbonyl}-2-isobutyl-5-(1,3-thiazol-2-yl)-2,5dihydro-1H-pyrrole-2-carboxylic acid;
rel-(2S,5R)-1-(3-bromo-4-tert-butylbenzoyl)-2-isobutyl-5-(1,3-thiazol-2-yl)-2,5-dihydro-1H-pyrrole-2,4-dicarboxylic acid;
rel-(2S,5R)-1-(3-bromo-4-tert-butylbenzoyl)-4-carbamoyl-2-isobutyl-5-(1,3-thiazol-2-yl)-2,5dihydro-1H-pyrrole-2-carboxylic acid;
and salts, solvates, esters and individual enantiomers thereof.

Also included in the present invention are pharmaceutically acceptable salt complexes. The present invention also covers the physiologically acceptable salts of the compounds of formula (I). Suitable physiologically acceptable salts of the compounds of formula (I) include acid salts, for example sodium, potassium, calcium, magnesium and tetraalkylammonium and the like, or mono- or di-basic salts with the appropriate acid for example organic carboxylic acids such as acetic, lactic, tartaric, malic, isethionic, lactobionic and succinic acids; organic sulfonic acids such as methanesulfonic, ethanesulfonic, benzenesulfonic and p-toluenesulfonic acids and inorganic acids such as hydrochloric, sulfuric, phosphoric and sulfamic acids and the like.

The present invention also relates to solvates of the compounds of Formula (I), for example hydrates.

The present invention also relates to pharmaceutically acceptable esters of the compounds of Formula (I), for example carboxylic acid esters —COOR, in which R is selected from straight or branched chain alkyl, for example n-propyl, n-butyl, alkoxyalkyl (e.g. methoxymethyl), aralkyl (e.g.benzyl), aryloxyalkyl (e.g. phenoxymethyl), aryl (e.g. phenyl optionally substituted by halogen, C₁₋₄alkyl or C₁₋₄alkoxy or amino). Unless otherwise specified, any alkyl moiety present in such esters preferably contains 1 to 18 carbon atoms, particularly 1 to 4 carbon atoms. Any aryl moiety present in such esters preferably comprises a phenyl group.

It will further be appreciated that certain compounds of the present invention may exist in different tautomeric forms. All tautomers are contemplated to be within the scope of the present invention.

Compounds of Formula (I) may be prepared by reaction of a compound of Formula (II) 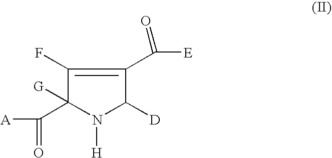
in which A, D, E, F and G are as defined above for Formula (I); with a suitable acylating agent, for example R³C(O)-hal, wherein hal is a halo atom, preferably chloro or bromo. Preferably the reaction is carried out in a suitable solvent, for example dichloromethane or chloroform, in the presence of a suitable base, for example triethylamine.

Compounds of Formula (II) may be prepared by reaction of a compound of Formula (III) 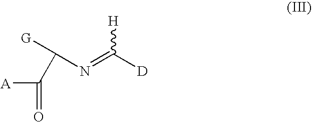
wherein A, D and G are as defined for Formula (I) above; with a compound of Formula (IV) 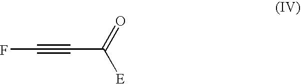
wherein E and F are as defined for Formula (I). Preferably, the reaction is carried out in a suitable solvent, for example TBF or acetonitrile, optionally in the presence of a Lewis acid catalyst, such as lithium bromide or silver acetate, and a base, such as triethylamine, 1,8-diazabicyclo[5,4,0]undec-7-ene (DBU) or tetramethyl guanidine. Alternatively, the reaction is carried out in a suitable solvent, for example THF or acetonitrile, in the presence of an acid, such as acetic acid, or the reaction may be carried out by heating compounds of Formula (III) and Formula (IV) in a suitable solvent, for example toluene, xylene or acetonitrile in the absence of a catalyst.

Compounds of Formula (III) and (IV) are known in the art or may be prepared by standard literature procedures.

It will be appreciated that racemic compounds of Formula (I) and (II) may be optionally resolved into their individual enantiomers. Such resolutions may conveniently be accomplished by standard methods known in the art For example, a racemic compound of

Formula (I) and (II) may be resolved by chiral preparative HPLC. Alternatively, racemic compounds of Formula (I) and (II) may be resolved by standard diastereoisomeric salt formation with a chiral acid or base reagent as appropriate. Such techniques are well established in the art.

With appropriate manipulation and protection of any chemical functionality, synthesis of the remaining compounds of Formula (I) is accomplished by methods analogous to those above and to those described in the Experimental section. Example protecting groups can be found, but are not restricted to, those found in T. W. Greene and P. G. M. Wuts ‘Protective Groups in Organic Synthesis’, 3^{rd} Ed (1999), J Wiley and Sons. It will be appreciated that with appropriate manipulation, a compound of Formula (I) may be interconverted to a compound of Formula (I) with a different D group by methods well known in the art.

### EXAMPLES

### Intermediate 1

### 2-[N-(1,3-Thiazol-2-yhnethylene)amino]-4-methyl-pentanoic acid, tert-butyl ester

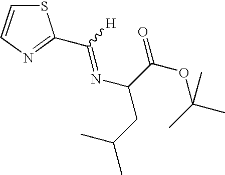

A stirred mixture of 2-amino-4-methyl-pentanoic acid tert-butyl ester, hydrochloride (5.00 g, 22.34 mmol), 1,3-thiazole-2-carboxaldehyde (2.53 g, 22.34 mmol) and triethylamine (3.10 mL, 22.3 mmol) in dichloromethane (60 mL) was heated under reflux under nitrogen for 19 hours. The reaction mixture was allowed to cool to room temperature, washed twice with water, dried over Na₂SO₄ and evaporated to give the title compound as an oil.

¹H NMR (CDCl₃): δ 8.46 (s, 1H), 7.94 (d, 1H), 7.44 (dd, 1H), 4.07 (dd, 1), 1.89-1.74 (m, 2H), 1.64-1.52 (m, 1H), 1.48 (s, 9H), 0.96 (d, 3H) and 0.90 (d, 3H).

### Intermediate 2

### rel-(2S,5R)-2-isobutyl-5-(1,3thiazol-2-yl)-2,5-dihydro-1H-pyrrole-2,4-dicarboxylic acid, 2-tert-butyl ester, 4-methyl ester

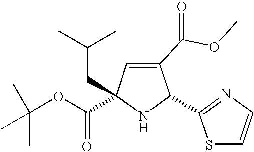

To a cooled (0° C.) stirred solution of Intermediate 1 (0.250 g, 0.885 mmol) in anhydrous THF (5 mL) under nitrogen, was added triethylamine (0.123 mL, 1 eq.) followed by lithium bromide (77 mg, 1 eq.) and methylpropiolate (0.08 mL, 1 eq.).

The mixture was stirred at 0° C. for 5 minutes, and then the ice/water bath was removed and stirring was continued at ambient temperature for 2 hours. Aqueous ammonium chloride was added with rapid stirring and the resulting mixture was extracted twice with ethyl acetate. The combined organic layers were dried over sodium sulphate and evaporated. The residue was purified by chromatography on silica gel using cyclohexane-ethyl acetate (8:2 v/v) as eluent to provide the title compound as an oil.

Mass spec m/z calcd for (C₁₈H₂₆N₂O₄S+H)⁺: 367 Mass spec (electrospray) Found: (M+H)⁺ 367.

### Intermediate 3

### rel-(2S,5R)-1-(4-tert-butylbenzoyl)-2-isobutyl-5-(1,3-thiazol-2-yl)-2,5-dihydro-1H-pyrrole-2,4-dicarboxylic acid, 2-tert-butyl ester, 4-methyl ester

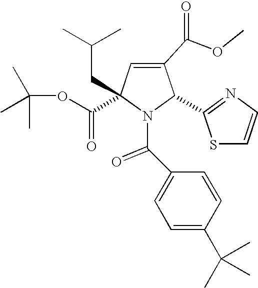

To a stirred solution of Intermediate 2 (0.137 mg, 0.37 mmol) in anhydrous dichloromethane (5 mL) was added triethylamine (0.064 mL, 0.46 mmol) and 4-tert-butylbenzoyl chloride (0.082 mL, 0.44 mmol). This mixture was stirred and heated under reflux for 18 hours. The mixture was then washed with water and extract with dichloromethane. The organic phase was dried (Na₂SO₄) and evaporated. The residue was purified by chromatography on silica gel using cyclohexane-ethyl acetate (6:1 v/v) as eluent to provide the title compound as a solid.

Mass spec m/z calcd for (C₂₉H₃₉N₂O₅S+H)⁺: 527. Mass spec (electrospray) Found: (M+H)⁺=527.

### Intermediate 4

### rel-(2S,5R)-1-(3bromo-4-tert-butylbenzoyl)-2-isobutyl-5(1,3thiazol-2-yl)-2,5-dihydro-1H-pyrrole-2,4-dicarboxylic acid, 2-butyl ester, 4-methyl ester

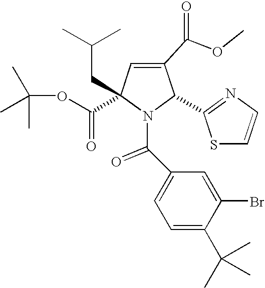

Prepared in a similar manner to that described in Intermediate 3 using Intermediate 2 and 3-bromo-4-tert-butylbenzoyl chloride. Chromatography on silica gel using dichloromethane-ethyl acetate (50:1 v/v) as eluent provided the title compound as a solid.

Mass spec m/z calcd for (C₂₉H₃₇BrN₂O₅S+H)⁺: 605/607 Mass spec (electrospray) Found: (M+H)⁺=605/607

### Intermediate 5

### rel-(2S,5R)-1-(4-tert-butylbenzoyl)-2-isobutyl-5-(1,3-thiazol-2-yl)-2,5-dihydro-1H-pyrrole-2,4-dicarboxylic acid, 2-tert-butyl ester

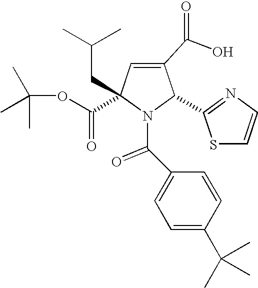

To a solution of Intermediate 3 (64.5 mg, 0.12 mmol) in methanol (4 mL) was added a solution of 2M sodium hydroxide (0.122 mL) and the resulting mixture was stirred and heated under reflux for 20 hours. The solvent was evaporated and the residue was dissolved in dichloromethane (5 mL), acidified with hydrochloric acid (2M) and extracted with water/dichloromethane. The organic phase was dried (Na₂SO₄) and evaporated to give the title compound as a solid.

Mass spec m/z calcd for (C₂₈H₃₆N₂O₅S+H)⁺: 513. Mass spec (electrospray) Found: (M+H)⁺=513.

### Intermediate 6

### rel(2S,5R)-1-(3-bromo-4-tert-butylbenzoyl)-2-isobutyl-5-(1,3-thiazol-2-yl)-2,5-dihydro-1H-pyrrole-2,4-dicarboxylic acid, 2-tert-butyl ester

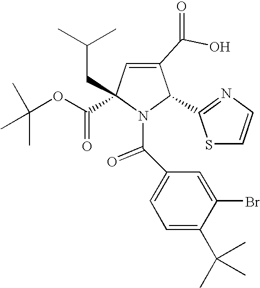

Prepared in a similar manner to that described for Intermediate 5 using Intermediate 4.

Mass spec m/z calcd for (C₂₈H₃₅BrN₂O₅S+H)⁺: 591/593 Mass spec (electrospray) Found (M+H)⁺=591/593

### Intermediate 7

### rel-(2S,5R)-2-isobutyl-3-methyl-5-(1,3-thiazol-2-yl)-2,5-dihydro-1H-pyrrole-2,4-dicarboxylic acid, 2-tert-butyl ester, 4-methyl ester

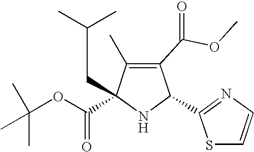

Intermediate 1 (500 mg, 1.77 mmol.) was dissolved in toluene (8 mL) and methyl-2-butynoate (0.18 ml, 1.77 mmol) was added. The solution was heated under reflux over 72 hours and was then quenched with a solution of saturated NH₄Cl (5 mL) and extracted with ethyl acetate. The combined organic layers were dried with Na₂SO₄ and concentrated. The residue was purified by chromatography on silica gel using cyclohexane-ethyl acetate (8:2 v/v) as eluent, to afford the title compound.

Mass spec m/z calc. for (C₁₉H₂₈N₂O₄S+H)⁺: 381 Mass spec (electrospray) Found (M+H)⁺: 381

### Intermediate 8

### rel-(2S,5R)-1-(4-tert-butylbenzoyl)-2-isobutyl-3-methyl-5-(1,3-thiazol-2-yl)-2,5-dihydro-1H-pyrrole-2,4-dicarboxylic acid, 2-tert-butyl ester, 4-methyl ester

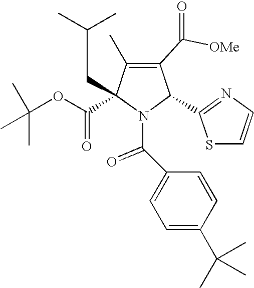

In a similar manner to that described in Intermediate 3 and using Intermediate 7, the title compound was prepared and purified by reverse phase HPLC (ABZ column, 10 cm×21.2 cm×5 um, solvent A: 950:50:0.05 acetonitrile:water:formic acid. solvent B: 0.1% aqueous formic acid. Gradient from 40% solvent A to 100% solvent A) and obtained as a solid.

Mass spec m/z calc. for (C₃₀H₄₀O₅N₂S+H)⁺: 541 Mass spec (electrospray) Found: (M+H)⁺: 541

### Intermediate 9

### rel-(2S,5R)-1-(4-tert-butylbenzoyl)-2-isobutyl-3-methyl-5-(1,3-thiazol)-2-yl)-2,5dihydro-1H-pyrrole-2,4-dicarboxylic acid, 2-tert-butyl ester

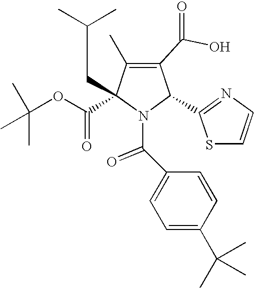

In a similar manner to that described for Intermediate 4 and using Intermediate 8, the title compound was prepared as a solid.

Mass spec m/z calc. for (C₂₉H₃₈O₅N₂S+H)⁺: 527 Mass spec (electrospray) Found: (M+H)⁺: 527

### Intermediate 10

### rel-(2S,5R)-1-(4-tert-butylbenzoyl)-4-carbamoyl-2-isobutyl-5-(1,3thiazol-2-yl)-2,5-dihydro-1H-pyrrole-2-carboxylic acid, 2-tert-butyl ester

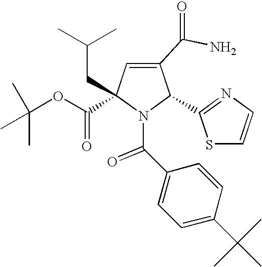

To a solution of Intermediate 5 (40 mg, 0.08 mmol) dissolved in DMF (dimethylformamide) (5 mL) was added DIPEA (diisopropylethylamine) (27 ml; 0.16 mmol, 2 eq), ammonium chloride (6 mg, 0.08 mmol) and HATU ([O-(7-azabenzotriazol-1-yl)-1,1,3,3-tetramethyluronium]hexafluorophosphate) coupling reagent (30 mg, 0.08 mol). The resulting mixture was stirred at room temperature overnight The reaction was quenched with a saturated solution of NaHCO₃ (15 mL) and extracted with water and ethyl acetate. The combined organic layers were dried (Na₂SO₄) and evaporated to give a crude compound that was purified by reverse phase HPLC (ABZ column, 10 cm×21.2 cm×5 um, solvent A: 950:50:0.05 acetonitrile:water:formic acid. solvent B: 0.1% aqueous formic acid. Gradient from 40% solvent A to 100% solvent A) thus affording the title compound.

Mass spec m/z calc. for (C₂₈H₃₇N₃O₄S+H)⁺: 512 Mass spec (electrospray) Found (M+H)⁺: 512.

Intermediates 11-17 were prepared in a similar manner from Intermediate 5 using the appropriate reagent.

### Intermediate 11

### rel-(2S,5R)-1-(4-tert-butylbenzoyl)-4-{[(3-tert-butoxycarbonylethyl)amino]carbonyl}-2-isobutyl-5-(1,3-thiazol-2-yl)-2,5-dihydro-1H-pyrrole-2-carboxylic acid, 2tert-butyl ester

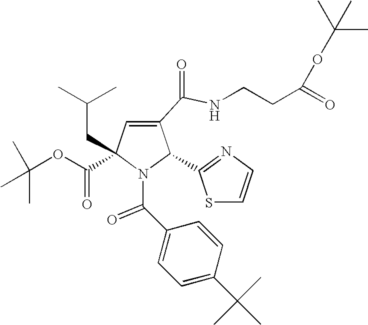

Mass spec m/z calc. for (C₃₅H₄₉N₃O₆S+H)⁺: 640 Mass spec (electrospray) Found (M+H)⁺: 640.

### Intermediate 12

### rel-(2S,5R)-1-(4-tert-butylbenzoyl)-4-{[(3-carboxamidoethyl)amino]carbonyl}-2-isobutyl-5-(1,3-thiazol-2-yl)-2,5dihydro-1H-pyrrole-2-carboxylic acid, 2-tert-butyl ester

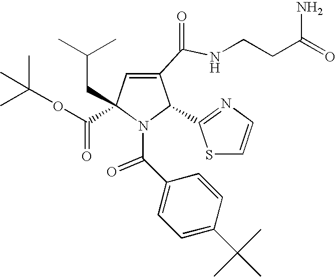

Mass spec m/z calc. for (C₃₁H₄₂N₄O₅S+H)⁺: 583 Mass spec (electrospray) Found (M+H)⁺: 583

### Intermediate 13

### rel-(2S,5R)-1-(4tert-butylbenzoyl)-4-}[(2-tert-butoxycarbonylmethyl)amino]carbonyl}-2-isobutyl-5-(1,3thiazol-2-yl)-2,5dihydro-1H-pyrrole-2-carboxylic acid, 2-tert-butyl ester

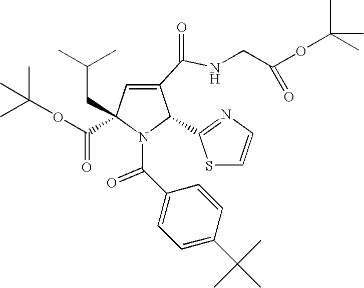

Mass spec m/z calc. for (C₃₄H₄₇N₃O₆S+H)⁺: 626 Mass spec (electrospray) Found (M+H)⁺: 626.

### Intermediate 14

### rel-(2S,5R)-1-(4-tert-butylbenzoyl)-4-[(isobutylamino)carbonyl]-2-isobutyl-5-(1,3-thiazol-2-yl)-2,5-dihydro-1H-pyrrole-2-carboxylic acid, 2-tert-butyl ester

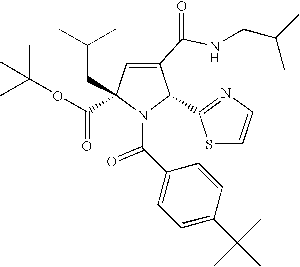

Mass spec m/z calc. for (C₃₂H₄₅N₃O₄S+H)₊: 568 Mass spec (electrospray) Found (M+H)⁺: 568.

### Intermediate 15

### rel-(2S,5R)-1-(4-tert-butylbenzoyl)-4-[(benzylamino)carbonyl]-2-isobutyl-5-(1,3-thiazol-2-yl)-2,5-dihydro-1H-pyrrole-2-carboxylic acid, 2-tert-butyl ester

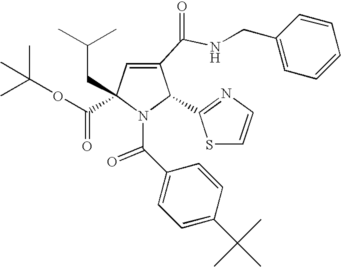

Mass spec m/z calc. for (C₃₅H₄₃N₃O₄S+H)⁺: 602 Mass spec (electrospray) Found (M+H)⁺: 602

### Intermediate 16

### rel-(2S,5R)-1-(4-tert-butylbenzoyl)-4-{[(cyclohexylmethyl)amino]carbonyl}-2-isobutyl-5-(1,3-thiazol-2-yl)-2,5-dihydro-1H-pyrrole-2-carboxylic acid, 2-tert-butyl ester

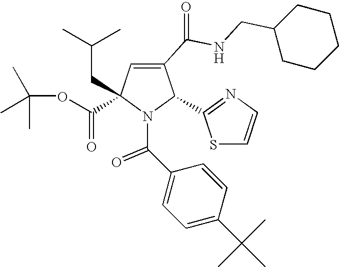

Mass spec m/z calc. for (C₃₅H₄₉N₃O₄S+H)⁺: 608 Mass spec (electrospray) Found (M+H)⁺: 608.

### Intermediate 17

### rel-(2S, 5)-1-(4-butylbenzoyl)-4-}[(cyanomethyl)amino]carbonyl}-2-isobutyl-5-(1,3-thiazol-2-yl)-2,5-dihydro-1H-pyrrole-2-carboxylic acid, 2-tert-butyl ester

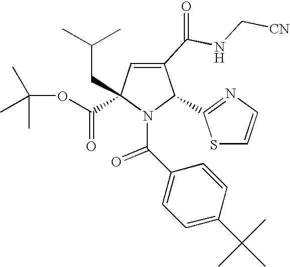

Mass spec m/z calc. for (C₃₀H₃₈N₄O₄S+H)⁺: 551 Mass spec (electrospray) Found (M+H)⁺: 551.

### Intermediate 18

### rel-(2S,5R)-1-(3bromo-4-tert-butylbenzoyl)-4-carbamoyl-2-isobutyl-5-(1,3-thiazol-2-yl)-2,5-dihydro-1H-pyrrole-2,4dicarboxylic acid, 2-tert-butyl ester

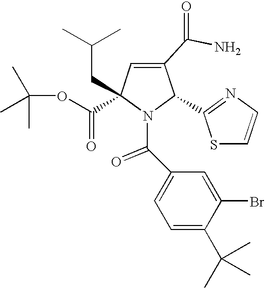

Prepared in a similar manner to that described for Intermediate 10, from Intermediate 6.

Mass spec m/z calc. for (C₂₈H₃₆BrN₃O₄S+H)⁺: 590/592 Mass spec (electrospray) Found (M+H)⁺: 590/592.

### Example 1

### rel-(2S,5R)-1-(4-tert-butylbenzoyl)-2-isobutyl-5-(1,3-thiazol-2-yl)-2,5-dihydro-1H-pyrrole-2,4dicarboxylic acid

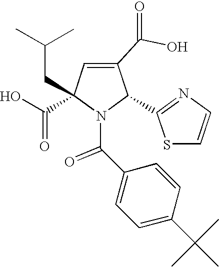

To a solution of Intermediate 5 (44 mg, 8.85 mmol) in dichloromethane (5 mL) was added trifluoroacetic acid (1 mL) and the solution stirred at ambient temperature overnight The reaction mixture was evaporated and the residue triturated with diethylether to give the title compound as a solid.

Mass spec m/z calc for (C₂₄H₂₈N₂O₅S+H)⁺: 457 Mass spec (electrospray) Found: (M+H)⁺457

### Example 2

### rel-(2S,5R)-1-(4-tert-butylbenzoyl)-2-isobutyl-3-methyl-5-(1,3-thiazol-2-yl)-2,5-dihydro-1H-pyrrole-2,4-dicarboxylic acid

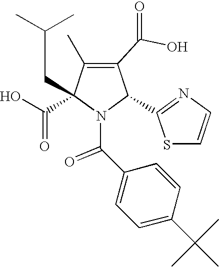

Using Intermediate 9 and in a similar manner to that described in Example 1, the title compound was prepared as a solid.

Mass spec m/z calc. for (C₂₅H₃₀N₂O₅S+H)⁺: 471 Mass spec (electrospray) Found: (M+H)⁺: 471

### Example 3

### rel-(2S,5R)-1-(4tert-butylbenzoyl)-4-carbamoyl-2-isobutyl-5-(1,3-thiazol-2-yl)-2,5-dihydro-1H-pyrrole-2-carboxylic acid

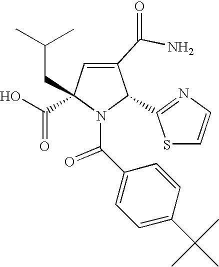

Using Intermediate 10 and in a similar manner to that described in Example 1, the title compound was prepared as a solid.

Mass Spec m/z calc for (C₂₄H₂₉N₃O₄S+H)⁺: 456 Mass spec (electrospray) Found (M+H)⁺: 456.

### Example 4

### rel-(2S,5R)-1-(4-tert-butylbenzoyl)-4-{[(2-carboxyethyl)amino]carbonyl}-2-isobutyl-5-(1,3-thiazol-2-yl)-2,5-dihydro-1H-pyrrole-2-carboxylic acid

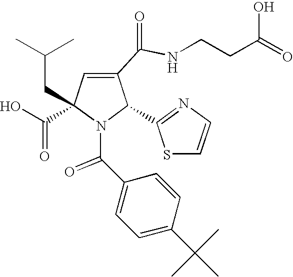

A solution of Intermediate 11 (45 mg) in dichloromethane (1 mL) was treated with trifluoroacetic acid (1 mL) and placed at room temperature for 5 hours. Volatile materials were removed and the residue was triturated with ether to give the title compound as a solid.

Mass spec m/z calc for (C₂₇H₃₃N₃O₆S+H)⁺: 528 Mass spec (electrospray) Found (M+H)⁺: 528.

Examples 5-12 were prepared in a similar manner.

### Example 5

### rel-(2S,5R)-1-(4-tert-butylbenzoyl)-4-{[(3-carboxamidoethyl)amino]carbonyl}-2-isobutyl-5-(1,3-thiazol-2-yl)-2,5-dihydro-1H-pyrrole-2-carboxylic acid

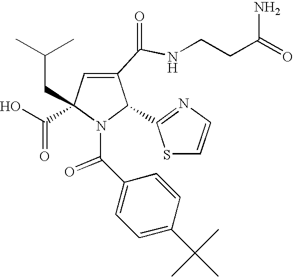

Prepared from Intermediate 12.

Mass spec m/z calc for (C₂₇H₃₄N₄O₅S+H)⁺: 527 Mass spec (electrospray) Found (+H)⁺: 527.

### Example 6

### rel-(2S,5S)-1-(4-tert-butylbenzoyl)-4-{[(2-carboxymethyl)amino]carbonyl}-2-isobutyl-5-(1,3-thiazol-2-yl)-2,5-dihydro-1H-pyrrole-2-carboxylic acid

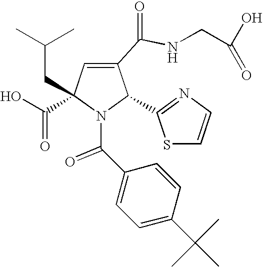

Prepared from Intermediate 13.

Mass spec m/z calc for (C₂₆H₃₁N₃O₆S+H)⁺: 514. Mass spec (electrospray) Found (M+H)⁺: 514.

### Example 7

### rel-(2S,5R)-1-(4-tert-butylbenzoyl)-4-[isobutylamino)carbonyl]-2-isobutyl-5-(1,3-thiazol-2yl)-2,5-dihydro-1H-pyrrole-2-carboxylic acid

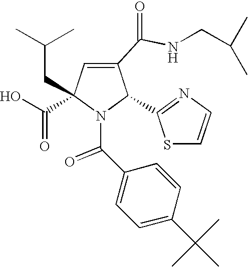

Prepared from Intermediate 14.

Mass spec m/z calc for (C₂₈H₃₇N₃O₄S+H)⁺: 512. Mass spec (electrospray) Found (M+H)⁺: 512.

### Example 8

### rel-(2S,5R)-1-(4-tert-butylbenzoyl)-4-[(benzylamino)carbonyl]-2-isobutyl-5-(1,3-thiazol-2-yl)-2,5-dihydro-1H-pyrrole-2-carboxylic acid

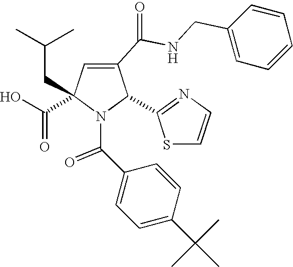

Prepared from Intermediate 15.

Mass spec m/z calc for (C₃₁H₃₅N₃O₄S+H)⁺: 546 Mass spec (electrospray) Found (M+H)⁺: 546

### Example 9

### rel-(2S,5R)-1-(4tert-butylbenzoyl)-4-{[(cyclohexylmethyl)amino]carbonyl}-2-isobutyl-5-(1,3-thiazol-2-yl)-2,5-dihydro-1H-pyrrole-2-carboxylic acid

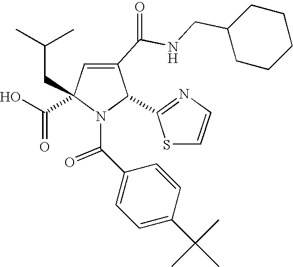

Prepared from Intermediate 16.

Mass spec m/z calc for (C₃₁H₄₁N₃O₄S+H)⁺: 552 Mass spec (electrospray) Found (M+H)⁺: 552

### Example 10

### rel-(2S,5R)-1-(4-tert-butylbenzoyl)-4-{[(cyanomethyl)amino]carbonyl}-2-isobutyl-5-(1,3-thiazol-2-yl)-2,5-dihydro-1H-pyrrole-2-carboxylic acid

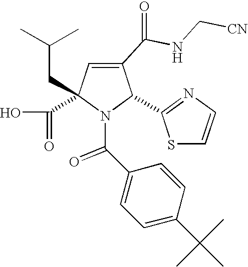

Prepared from Intermediate 17.

Mass spec m/z calc for (C₂₆H₃₀N₄O₄S+H)⁺: 495 Mass spec (electrospray) Found (M+H)⁺: 495

### Example 11

### rel-(2S,5R)-1-(3-bromo-4-tert-butylbenzoyl)-2-isobutyl-5-(1,3-thiazol-2-yl)-2,5-dihydro-1H-pyrrole-2,4dicarboxylic acid

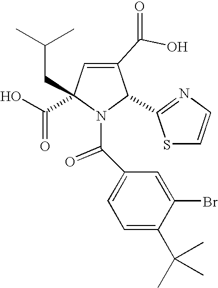

Prepared from Intermediate 6.

Mass spec m/z calc for (C₂₄H₂₇BrN₂O₅S+H)⁺: 535/537 Mass spec (electrospray) Found (M+H)⁺: 535/537

### Example 12

### rel-(2S,5R)-1-(3-bromo-4-tert-butylbenzoyl)-4-carbamoyl-2-isobutyl-5-(1,3-thiazol-2-yl)-2,5-dihydro-1H-pyrrole-2-carboxylic acid

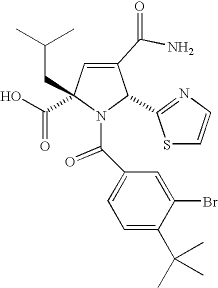

Prepared from Intermediate 18.

Mass spec m/z calc for (C₂₄H₂₈BrN₃O₄S+H)⁺: 534/536 Mass spec (electrospray) Found (M+H)⁺: 534/536

The compounds according to the invention may be formulated for administration in any convenient way, and the invention therefore also includes within its scope pharmaceutical compositions for use in therapy, comprising a compound of formula (I) or a physiologically acceptable salt or solvate thereof in admixture with one or more physiologically acceptable diluents or carriers.

The compounds of the present invention can be administered by different routes including intravenous, intraperitoneal, subcutaneous, intramuscular, oral, topical, transdermal, or transmucosal administration. For systemic administration, oral administration is preferred. For oral administration, for example, the compounds can be formulated into conventional oral dosage forms such as capsules, tablets and liquid preparations such as syrups, elixirs and concentrated drops.

Alternatively, injection (parenteral administration) may be used, e.g., intramuscular, intravenous, intraperitoneal, and subcutaneous. For injection, the compounds of the invention are formulated in liquid solutions, preferably, in physiologically compatible buffers or solutions, such as saline solution, Hank's solution, or Ringer's solution. In addition, the compounds may be formulated in solid form and redissolved or suspended immediately prior to use. Lyophilized forms can also be produced.

Systemic administration can also be by transmucosal or transdermal means. For transmucosal or transdermal administration, penetrants appropriate to the barrier to be permeated are used in the formulation. Such penetrants are generally known in the art and include, for example, for transmucosal administration, bile salts and fusidic acid derivatives. In addition, detergents may be used to facilitate permeation. Transmucosal administration, for example, may be through nasal sprays, rectal suppositories, or vaginal suppositories.

For topical administration, the compounds of the invention can be formulated into ointments, salves, gels, or creams, as is generally known in the art.

The amounts of various compounds to be administered can be determined by standard procedures taking into account factors such as the compound (IC₅₀) potency, (EC₅₀) efficacy, and the biological half-life (of the compound), the age, size and weight of the patient, and the disease or disorder associated with the patient The importance of these and other factors to be considered are known to those of ordinary skill in the art

Amounts administered also depend on the routes of administration and the degree of oral bioavailability. For example, for compounds with low oral bioavailability, relatively higher doses will have to be administered. Oral administration is a preferred method of administration of the present compounds.

Preferably the composition is in unit dosage form. For oral application, for example, a tablet, or capsule may be administered, for nasal application, a metered aerosol dose may be administered, for transdermal application, a topical formulation or patch may be administered and for transmucosal delivery, a buccal patch may be administered. In each case, dosing is such that the patient may administer a single dose.

Each dosage unit for oral administration contains suitably from 0.01 to 500 mg/Kg, and preferably from 0.1 to 50 mg/Kg, of a compound of Formula (I) or a pharmaceutically acceptable salt thereof, calculated as the free base. The daily dosage for parenteral, nasal, oral inhalation, transmucosal or transdermal routes contains suitably from 0.01 mg to 100 mg/Kg, of a compound of Formula(I). A topical formulation contains suitably 0.01 to 5.0% of a compound of Formula (I). The active ingredient may be administered from 1 to 6 times per day, preferably once, sufficient to exhibit the desired activity, as is readily apparent to one skilled in the art.

Composition of Formula (I) and their pharmaceutically acceptable salts which are active when given orally can be formulated as syrups, tablets, capsules and lozenges. A syrup formulation will generally consist of a suspension or solution of the compound or salt in a liquid carrier for example, ethanol, peanut oil, olive oil, glycerine or water with a flavoring or coloring agent. Where the composition is in the form of a tablet, any pharmaceutical carrier routinely used for preparing solid formulations may be used. Examples of such carriers include magnesium stearate, terra alba, talc, gelatin, acacia, stearic acid, starch, lactose and sucrose. Where the composition is in the form of a capsule, any routine encapsulation is suitable, for example using the aforementioned carriers in a hard gelatin capsule shell. Where the composition is in the form of a soft gelatin shell capsule any pharmaceutical carrier routinely used for preparing dispersions or suspensions may be considered, for example aqueous gums, celluloses, silicates or oils, and are incorporated in a soft gelatin capsule shell.

Typical parenteral compositions consist of a solution or suspension of a compound or salt in a sterile aqueous or non-aqueous carrier optionally containing a parenterally acceptable oil, for example polyethylene glycol, polyvinylpyrrolidone, lecithin, arachis oil or sesame oil.

Typical compositions for inhalation are in the form of a solution, suspension or emulsion that may be administered as a dry powder or in the form of an aerosol using a conventional propellant such as dichlorodifluoromethane or trichlorofluoromethane.

A typical suppository formulation comprises a compound of Formula (I) or a pharmaceutically acceptable salt thereof which is active when administered in this way, with a binding and/or lubricating agent, for example polymeric glycols, gelatins, cocoa-butter or other low melting vegetable waxes or fats or their synthetic analogs.

Typical dermal and transdermal formulations comprise a conventional aqueous or non-aqueous vehicle, for example a cream, ointment, lotion or paste or are in the form of a medicated plaster, patch or membrane.

No unacceptable toxological effects are expected when compounds of the present invention are administered in accordance with the present invention.

### Assay

The potential for compounds of the invention to inhibit NS5B wildtype HCV polymerase activity may be demonstrated, for example, using the following in vitro assay:

### In Vitro Detection of Inhibitors of HCV RNA-Dependent RNA Polymerase Activity

Incorporation of [³H]-UMP into RNA was followed by absorption of the RNA polymer onto a DEAE glass fibre filter. A synthetic template consisting of 16 mer oligoU hybridised to polyrA (10:1 w/w) was used as a homopolymer substrate.

Reaction Conditions were 22 μM [³H]-UTP (0.75 Ci/mmol), 1 mM-Dithiothreitol, 3.2 mM-MgCl₂, 20 mM-Tris-HCl, pH 7.0, 10 μg/ml polyA-oligoU, and 90 mM-NaCl. Note that 50 mM-NaCl is added with the enzyme.

HCV RNA Polymerase (Recombinant full-length NS5B (Lohmann et al, J. Virol. 71 (11), 1997, 8416 ‘Biochemical properties of hepatitis C virus NS5B RNA-dependent RNA polymerase and identification of amino acid sequence motifs essential for enzymatic activity’) expressed in baculovirus and purified to homogeneity) was diluted to about 50 μg protein/mL (dependent on specific activity) in 50 mM-Hepes, pH 7.0, 0.5M-NaCl, 20%-Glycerol, 0.05%-Triton X-100, 5 mM-Dithiothreitol, 0.1 mM-EDTA.

5× Concentrated Buffer minx was prepared using 1M-Tris-HCl (pH 7.0, 1 mL), 1M-MgCl₂ (0.16 mL), 1M-Dithiothreitol (0.05 mL), 5M-NaCl (0.4 mL), and Water (8.4 mL), Total 10 mL.

Substrate Mix was prepared using 5× Concentrated Buffer mix (12 μL), [³H]-UTP (1 μCi/μL; 21.7 μM, 1 μL), 22 μM-UTP (100 μM, 13.2 μL), 10 μg/mL polyA-oligoU (100 μg/mL, 6 μL), and Water (12.8 μL), Total 45 μL.

The Assay was set up using Substrate Mix (45 μL), compound (10 μL), and Diluted Enzyme (added last to start reaction) (5 μL), Total 60 μL. The reaction was performed in a U-bottomed, clear, 96-well plate. The reaction was mixed on a plate-shaker, after addition of the Enzyme, and incubated for 2 h at 22° C. After this time, the reaction was stopped by addition of 25 μL of 100 mM-EDTA.

A DEAE Filtermat (Part No. 1205-405 from Pharmacia) was pre-washed in water and alcohol and dried. 2×20 μL of the Stopped Assay Mix was spotted onto a square of the DEAE Filtermat. The DEAE Filtermat was washed for 2×5 min in SSC buffer (0.3M-NaCl, 30 mM-Na Citrate) followed by 2×2 min in water and 1×1 min in alcohol. The Filtermat was dried and sealed in a bag together with 10 mL of OptiScint HiSafe scintillation fluid. The radioactivity present on the filtermat was detected by scintillation counting on a Wallac 1205 Betaplate counter. After subtraction of background levels without enzyme, any reduction in the amount of radioactivity incorporated in the presence of a compound, compared to that in the absence, was taken as a measure of the level of inhibition. Ten concentrations of compounds were tested in two- or threefold dilutions. From the counts, percentage of inhibition at highest concentration tested or IC₅₀s for the compounds were calculated using Grafit3 or Grafit4 software packages.

The exemplified compounds had an IC₅₀ of <50 μm. Accordingly, the compounds of the invention are of potential therapeutic benefit in the treatment and prophylaxis of HCV. Preferred compounds had an IC₅₀ of <5 μM.

Thus, there is provided as a further aspect of the present invention a compound of formula (I) or a physiologically acceptable salt or solvate thereof for use in human or veterinary medical therapy, particularly use in the treatment and/or prophylaxis of a viral infection, particularly HCV infection.

It will be appreciated that reference herein to treatment includes, but is not limited to prevention, retardation, prophylaxis, therapy and cure of the disease. It will further be appreciated that references herein to treatment or prophylaxis of HCV infection includes treatment or prophylaxis of HCV-associated disease such as liver fibrosis, cirrhosis and hepatocellular carcinoma.

According to another aspect of the invention, there is provided the use of a compound of formula (I) or a physiologically acceptable salt or solvate thereof for the manufacture of a medicament for the treatment and/or prophylaxis of viral infection, particularly HCV infection.

In a further or alternative aspect there is provided a method for the treatment of a human or animal subject with viral infection, particularly HCV infection, which method comprises administering to said human or animal subject an effective amount of a compound of formula (I) or a physiologically acceptable salt or solvate thereof.

The pharmaceutical compositions according to the invention may also be used in combination with other therapeutic agents, for example immune therapies (eg. interferon), therapeutic vaccines, antifibrotic agents, anti-inflammatory agents such as corticosteroids or NSAIDs, bronchodilators such as beta-2 adrenergic agonists and xanthines (e.g. theophylline), mucolytic agents, anti-muscarinics, anti-leukotrienes, inhibitors of cell adhesion (e.g. ICAM antagonists), anti-oxidants (eg N-acetylcysteine), cytoline agonists, cytokine antagonists, lung surfactants and/or antimicrobial and anti-viral agents (eg ribavirin and amantidine). The compositions according to the invention may also be used in combination with gene replacement therapy.

The invention thus provides, in a further aspect, a combination comprising a compound of formula (I) or a physiologically acceptable salt or solvate thereof together with another therapeutically active agent.

The combinations referred to above may conveniently be presented for use in the form of a pharmaceutical formulation and thus pharmaceutical formulations comprising a combination as defined above together with a pharmaceutically acceptable carrier thereof represent a further aspect of the invention. The individual components of such combinations may be administered either sequentially or simultaneously in separate or combined pharmaceutical formulations. Appropriate doses of known therapeutic agents will be readily appreciated by those skilled in the art.

All publications, including but not limited to patents and patent applications cited in this specification are herein incorporated by reference as if each individual publication were specifically and individually indicated to be incorporated by reference as though fully set forth.

1. A compound of formula (I) 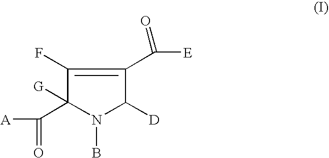
wherein:
A represents OR¹, NR¹R², or R¹ wherein R¹ and R² are independently selected from the group consisting of hydrogen, C₁₋₆alkyl, aryl, heteroaryl, arylalkyl, and heteroarylalkyl; or R¹ and R² together with the nitrogen atom to which they are attached form a 5 or 6 membered saturated cyclic group;
B represents C(O)R³ wherein R³ is selected from the group consisting of C₁₋₆alkyl, aryl, heteroaryl, arylalkyl, and heteroarylalkyl;
D represents C₁₋₆alkyl, aryl, heteroaryl or heterocyclyl;
E represents OR¹, NR¹R², or R¹ wherein R¹ and R² are independently selected from the group consisting of hydrogen, C₁₋₆alkyl, aryl, heteroaryl, arylalkyl or heteroarylalkyl; or R¹ and R² together with the nitrogen atom to which they are attached form a 5 or 6 membered saturated cyclic group;
F represents hydrogen, C₁₋₆alkyl, aryl or heteroaryl; and
G represents hydrogen, C₁₋₆alkyl, heterocyclylalkyl, arylalkyl or heteroarylalkyl; and salts, solvates and esters thereof, provided that when A is OR¹ then R¹ is other than tert-butyl.

2. A compound of Formula (I) as claimed in claim 1 selected from the group consisting of:
rel-(2S,5R)-1-(4-tert-butylbenzoyl)-2-isobutyl-5-(1,3-thiazol-2-yl)-2,5-dihydro-1H-pyyrole-2,4-dicarboxylic acid;
rel-(2S,5R)-1-(4-tert-butylbenzoyl)-2-isobutyl-3-methyl-5-(1,3-thiazol-2-yl)-2,5-dihydro-1H-pyrrole-2,4-dicarboxylic acid;
rel-(2S,5R)-1-(4-tert-butylbenzoyl)-4-carbamoyl-2-isobutyl-5-(1,3-thiazol-2-yl)-2,5-dihydro-1H-pyrrole-2-carboxylic acid;
rel-(2S,5R)-1-(4-tert-butylbenzoyl)-4-{[(2-carboxyethyl)amino]carbonyl}-2-isobutyl-5-(1,3-thiazol-2-yl)-2,5-dihydro-1H-pyrrole-2-carboxylic acid;
rel-(2S,5R)-1-(4-tert-butylbenzoyl)-4-{[(3-carboxamidoethyl)amino]carbonyl}-2-isobutyl-5-(1,3-thiazol-2-yl)-2,5-dihydro-1H-pyrrole-2-carboxylic acid;
rel-(2S,5R)-1-(4-tert-butylbenzoyl)-4{[(2-carboxymethyl)amino]carbonyl}-2-isobutyl-5-(1,3-thiazol-2-yl)-2,5-dihydro-1H-pyrrole-2-carboxylic acid;
rel-(2S,5R)-1-(4-tert-butylbenzoyl)-4-[(isobutylamino)carbonyl]-2-isobutyl-5-(1,3-thiazol-2-yl)-2,5-dihydro-1H-pyrrole-2-carboxylic acid;
rel-(2S,5R)-1-(4-tert-butylbenzoyl)-4-[(benzylamino)carbonyl]-2-isobutyl-5-(1,3-thiazol-2-yl)-2,5-dihydro-1H-pyrrole-2-carboxylic acid;
rel-(2S,5R)-1-(4-tert-butylbenzoyl)-4-{[(cyclohexylmethyl)amino]carbonyl}-2-isobutyl-5-(1,3-thiazol-2-yl)-2,5-dihydro-1H-pyrrole-2-carboxylic acid;
rel-(2S,5R)-1-(4-tert-butylbenzoyl)-4-{[(cyanomethyl)amino]carbonyl}-2-isobutyl-5-(1,3-thiazol-2-yl)-2,5-dihydro-1H-pyrrole-2-carboxylic acid;
rel-(2S,5R)-1-(3-bromo-4-tert-butylbenzoyl)-2-isobutyl-5-(1,3-thiazol-2-yl)-2,5-dihydro-1H-pyrrole-2,4-dicarboxylic acid; and
rel-(2S,5R)-1-(3-bromo-4-tert-butylbenzoyl)-4-carbamoyl-2-isobutyl-5-(1,3-thiazol-2-yl)-2,5-dihydro-1H-pyrrole-2-carboxylic acid;
salts, solvates, esters and individual enantiomers thereof.

3. A pharmaceutical formulation comprising a compound of Formula (I) as claimed in claim 1 in conjunction with a pharmaceutically acceptable diluent or carrier therefor.

4. A method of treating or preventing viral infection which comprises administering to a subject in need thereof, an effective amount of a compound as claimed in claim 1.

5. A method as claimed in claim 4 which involves inhibiting HCV.

6. A method as claimed in claim 4 in which the compound is administered in oral dosage form.

7. (canceled)

8. (canceled)

9. (canceled)

10. (canceled)

11. (canceled)

12. A process for the preparation of a compound of Formula (I) as claimed in claim 1, comprising reaction of a compound of Formula (II) 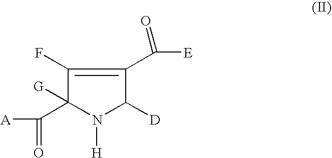
in which A, D, E, F and G are as defined above for Formula (I); with an acylating agent.

13. A compound as claimed in claim 1, wherein B represents C(O)R³ and R³ represents phenyl substituted in the para-position by tert-butyl and optionally further substituted by methyl, ethyl, methoxy, ethoxy, or halo.
